(19) United States
(12) Patent Application Publication
(10) Pub. No.: US 2019/0174727 A1
(43) Pub. Date: Jun. 13, 2019

(54) COLLAPSIBLE BEEHIVE ASSEMBLY

(71) Applicant: Joan Spiegel, Brookline, MA (US)

(72) Inventor: Joan Spiegel, Brookline, MA (US)

(21) Appl. No.: 16/218,408

(22) Filed: Dec. 12, 2018

Related U.S. Application Data
(60) Provisional application No. 62/597,620, filed on Dec. 12, 2017.

(51) Int. Cl. A01K 47/02; A01K 13/00

(52) U.S. Cl. CPC A01K 47/02; A01K 67/00; A01K 13/00

(57) ABSTRACT

A collapsible beehive assembly is comprised of a bottom assembly having a plurality of collapsible boxes stacked thereon. Each of the plurality of collapsible boxes is configured to house a plurality of beehive frames. Each of the plurality of collapsible boxes is further configured to receive one or more insulation components to winterize the collapsible beehive assembly. An inner cover is positioned on top of the plurality of collapsible boxes and a hive cover positioned on top of the inner cover to enclose the beehive frames within the collapsible boxes. A weight is positioned on top of the hive cover to retain the hive cover in a suitable position.

### CROSS-REFERENCE TO RELATED APPLICATIONS

The present application claims the priority benefit of U.S. Provisional Application No. 62/597,620 filed on Dec. 12, 2017, entitled “COLLAPSIBLE BEEHIVE ASSEMBLY”. The disclosure of the foregoing application is incorporated herein by reference in its entirety.

### FIELD

The present invention relates generally to beehive assemblies and more specially to collapsible and selectively insulated beehive assemblies for both personal and commercial use.

### BACKGROUND

Apiculture, otherwise known as beekeeping involves the maintenance of honeybee colonies for the collection of honey, beeswax, and pollen. Bees are also used as a pollinator of crops, or may be produced and sold to other beekeepers. This practice has been dated back many millennia as a valuable component of commerce. Man-made hives allow for continuity of a colony, rather than destroying the hive to harvest its products and starting over thereafter.

A variety of man-made hive designs have been constructed throughout history. In present day apiary, some notable designs include but are not limited to Langstroth hives, top-bar hives, long box hives, and Dartington long deep hives. Each hive design has its own advantages and disadvantages but share a common goal. Configurations largely depend on location and climate conditions.

Also, well known in the arts are modular beehive assemblies. These designs permit the beekeeper to select the overall size of the hive when in use, however they lack they advantage of a reduced storage size when not in use.

The current arts do not provide for a collapsible beehive assembly to facilitate storage and transportation.

### SUMMARY OF THE INVENTION

Embodiments disclosed herein relate to a collapsible beehive assembly is comprised of a bottom assembly having a plurality of collapsible boxes stacked thereon. Each of the plurality of collapsible boxes is configured to house a plurality of beehive frames. Each of the plurality of collapsible boxes is further configured to receive one or more insulation components to winterize the collapsible beehive assembly. An inner cover is positioned on top of the plurality of collapsible boxes and a hive cover positioned on top of the inner cover to enclose the beehive frames within the collapsible boxes. A weight is positioned on top of the hive cover to retain the hive cover in a suitable position.

In one aspect, each of the collapsible boxes is comprised of a first sidewall and a second sidewall. Each sidewall includes a handle portion to facilitate carrying of each of the collapsible boxes. A first end wall and a second end wall include a pivoting means permitting the folding of each of the end walls to a storage configuration.

In one aspect, each sidewall and each end wall are impermeable.

In one aspect, the storage configuration reduces a storage volume to between a tenth and a twentieth in relation to an in-use volume.

In one aspect, each of the sidewalls and each of the end walls slidingly engage with the one or more insulation components. The one or more insulation components are selected from the group consisting of Styrofoam, and tar paper.

In one aspect, the weight is comprised of an aperture and a closure wherein materials are disposed. The hive cover is comprised of a recessed portion dimensioned to receive and retain the weight. Materials of the weight may include water or sand.

In one aspect, the bottom assembly is further comprised of a varroa monitoring tray disposed within a varroa monitoring tray opening of the bottom assembly.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 illustrates an exploded view of the collapsible beehive assembly, according to some embodiments;

FIG. 2A illustrates a perspective view of the collapsible beehive assembly, according to some embodiments;

FIG. 2B illustrates a perspective view of the collapsible beehive assembly, according to some embodiments;

FIG. 3 illustrates a perspective view of the collapsible box in an in-use configuration, according to some embodiments;

FIG. 4A illustrates a top plan view of the collapsible box in a storage configuration, according to some embodiments;

FIG. 4B illustrates a perspective view of the collapsible box in a storage configuration, according to some embodiments;

FIG. 5 illustrates a perspective view of the collapsible box and insulation components, according to some embodiments;

FIG. 6 illustrates an exploded view of the collapsible box and the inner cover, according to some embodiments;

FIG. 7A illustrates an exploded view of the hive cover and weight, according to some embodiments;

FIG. 7B illustrates a perspective view of the bottom side of the hive cover, according to some embodiments; and

FIG. 8 illustrates perspective views of the bottom assembly and varroa monitoring tray, according to some embodiments.

### DETAILED DESCRIPTION

The specific details of the single embodiment or variety of embodiments described herein are set forth in this application. Any specific details of the embodiments are used for demonstration purposes only, and no unnecessary limitation or inferences are to be understood therefrom.

Before describing in detail exemplary embodiments, it is noted that the embodiments reside primarily in combinations of components related to the apparatus. Accordingly, the apparatus components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

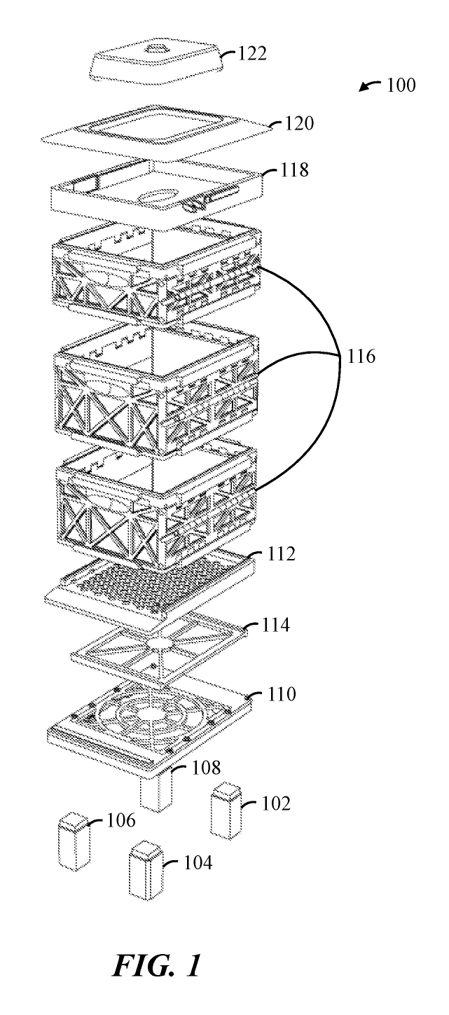

FIG. 1 illustrates the collapsible beehive assembly 100 according to an exemplary embodiment. A plurality of legs 102, 104, 106, 108 raise the collapsible beehive assembly 100 off of the ground surface and support each component of the collapsible beehive assembly 100. A bottom board assembly is comprised of a bottom assembly 110 and a top 112 having a varroa monitoring tray 114 disposed therebetween. A plurality of boxes 116 having varying dimensions are stacked atop the top 112 of the bottom board assembly. An inner cover 118 is positioned on top of the uppermost of the plurality of boxes 116 with a hive cover 120 positioned on top of the inner cover 118. A weight 122 is positioned on top of the hive cover 120 to hold the hive cover 120 in position. The collapsible beehive assembly 100 has impermeable sidewalls to adequately house and cultivate bees within the enclosure. The bottom of the hive I open permitting the ingress and egress of bees into and out of the environment wherein the collapsible beehive assembly 100 is disposed.

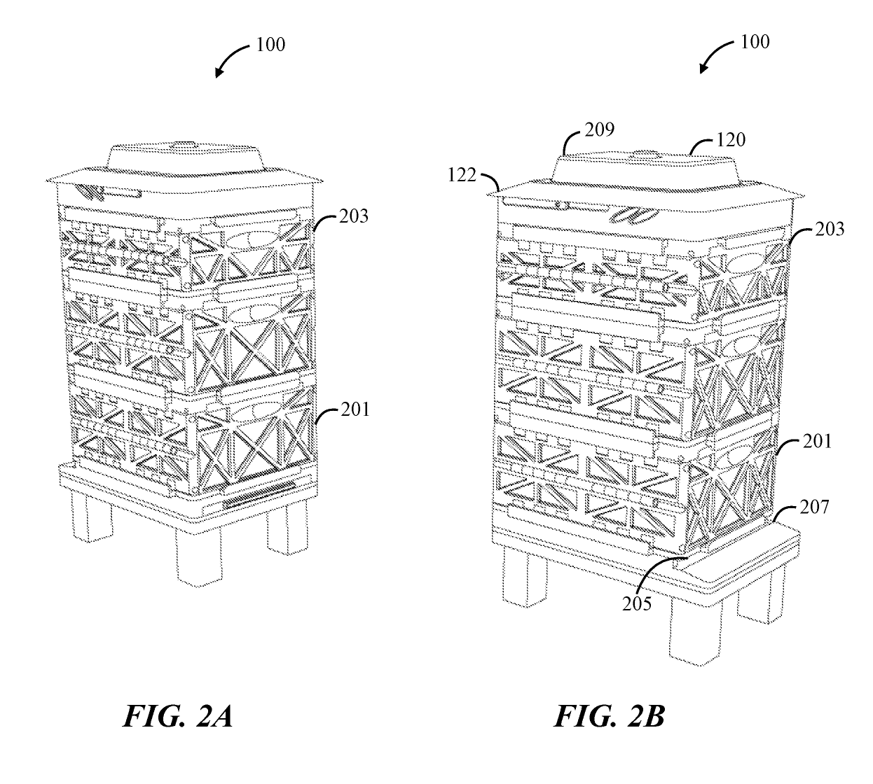

FIG. 2A and FIG. 2B illustrate the collapsible beehive assembly 100 in an in-use configuration. According to some embodiments, at least one large box 201 is positioned beneath a small box 203 such that each box is stacked atop one another. FIG. 2B shows a hive opening 205 positioned at the bottom end 207 which permits the ingress and egress of bees. The bottom end opposes the top end 209 having the weight 122 and hive cover 120.

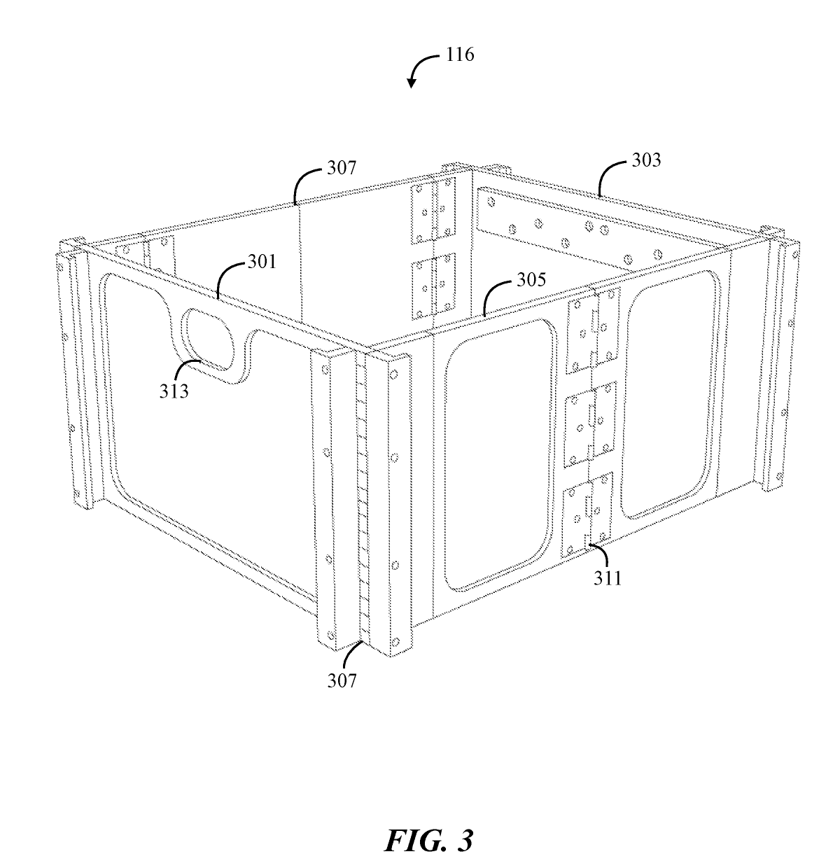

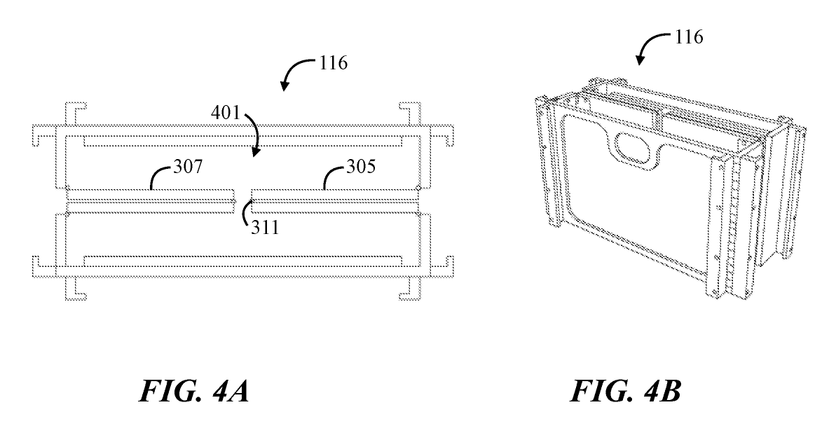

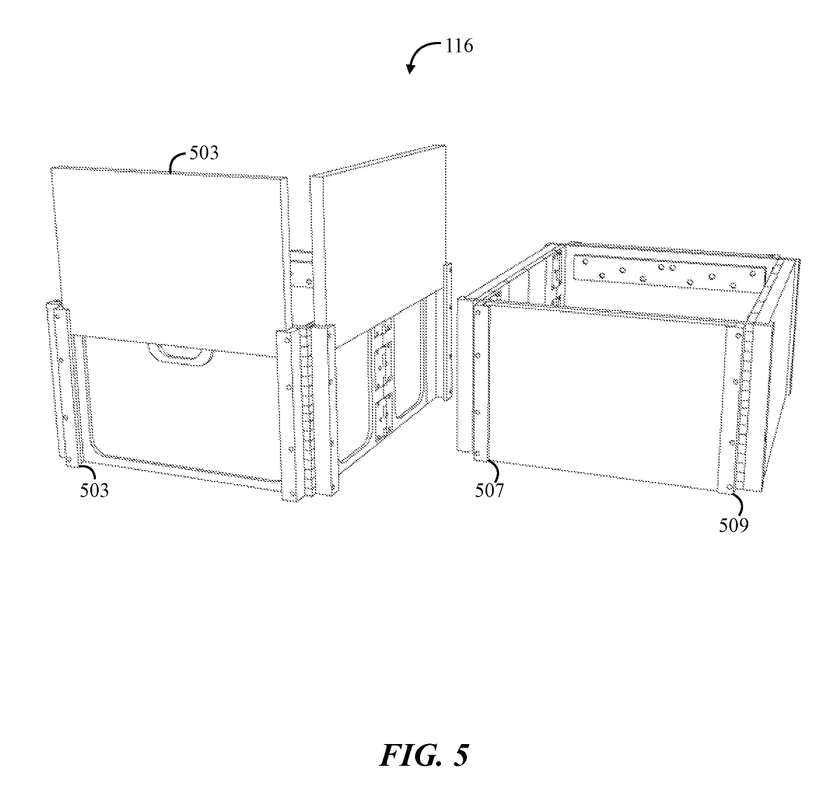

FIG. 3 illustrates a collapsible box 116 which forms one of the plurality of boxes 116 (three of which are shown in FIG. 1). The collapsible box 116 allows for convenient storage when the collapsible beehive assembly is not in use. During use, each collapsible box 116 along with the components described herein form a fully functional man-made beehive. Each collapsible box 116 has a first and a second of sidewall 301, 303 having equal dimensions and positioned on each side of the collapsible box 116. First and second end walls 305, 307 having equal dimensions are positioned opposite one another and adjacent to the first and second sidewalls 301, 303. Each sidewall 301, 303 and end wall 305, 307 are engaged to form a substantially rectangular container. Sidewalls 301, 303 and end walls 305, 307 may be comprised of a connection means 309 along each edge such that a substantially sealed connection is made when the collapsible box 116 is in an in-use configuration as shown in FIG. 3. Once in a storage configuration, as shown in FIG. 4A and FIG. 4B, the collapsible box 116 may be condensed to a total volume between a tenth and a twentieth of the total volume of the in-use configuration.

In some embodiments, and in further reference to FIG. 3, end walls 305, 307 have at least one pivoting means 311 allowing each end wall 305, 307 to pivot about the pivot means 311 to fold into the interior space of the interior cavity of the collapsible box 116. Meanwhile, each side wall 301, 303 travels toward one another to collapse into a configurations having a substantially decreased volume than the in-use configuration. Pivoting means 311 may include hinges, brackets, and other means known in the arts of collapsible containers. In a further embodiment, one or more locking mechanisms may be used to retain the assembly at least an in-use configuration wherein the end walls and sidewalls are erected, and potentially in the storage configuration when end walls and sidewalls are collapsed.

In some embodiments, the collapsible box 116 may fold from top to bottom, or side to side depending on the suitable configuration and ability to integrate with the various components described herein.

FIG. 4A and FIG. 4B illustrates the collapsible box 116 in a storage configuration wherein the interior cavity 401 has a substantially decreased volume. Each end wall 305, 307 are folded within the interior cavity 401 to increase storage capacity.

Referring back to FIG. 3, in some embodiments, each sidewall 301, 303 includes a handle portion 313 which may be recessed into the surface to allow a user to easily pick up and transport the collapsible box 116. One skilled in the arts may appreciate that handle portions 313 may be provided in a variety of configurations on the surface of the collapsible box 116.

Winterization is necessary for many beekeepers, and techniques may vary depending upon location and environment of the beehive. To ensure proper winterization, sidewalls 301, 303 and end walls 305, 307 may comprise a plurality of retainers 501 for an insulation component 503. In an embodiment, the retainer 501 may include pairs of protrusions 507, 509 disposed on the exterior of the sidewalls 301, 303 and end walls 305, 307. Each protrusion is configured to receive one or more pieces the insulation component 503 comprised of an insulated material such as Styrofoam, tar paper, or other means of insulation known in the arts such that the insulation is releasably engaged to accommodate changing temperatures which necessitate changes in insulation. In alternate embodiments, various forms of retainers 501 can be used to hold the insulation component 503 during use.

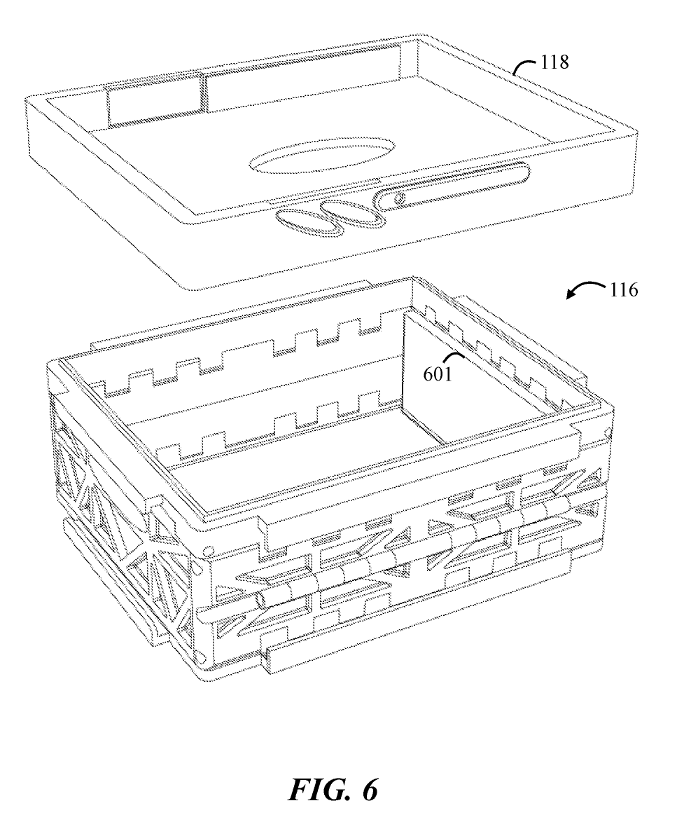

The collapsible beehive assembly, when in an in-use configuration as seen in FIG. 2A and FIG. 2B, includes an in-use interior cavity as shown in FIG. 3. The in-use interior cavity is dimensioned to house a plurality of frames permitting hives to be constructed by bees whom utilized the collapsible beehive assembly 100. As known in the arts, commonly a ledge 601 (see FIG. 6) is positioned on the interior of the sidewalls 301,303 and end walls 305,307 to retain the frames in position within the beehive assembly. Many styles of hives are utilized in the current state of the art but perhaps the most notable are Langstroth hives. One skilled in the art may appreciate that alternate hive style will also benefit from embodiments of the collapsible beehive assembly 100 in the instant application.

In a further embodiment, insulation components may be disposed along the bottom surface of the beehive assembly to provide further insulation. Much like the sidewall and end wall insulation, the bottom insulation component may be removably engaged with the bottom surface to provide modular insulation. In the present embodiment, the insulation component slidingly engages with the insulation retainers. Each insulation component may have a recessed portion to aid in carrying of the assembly.

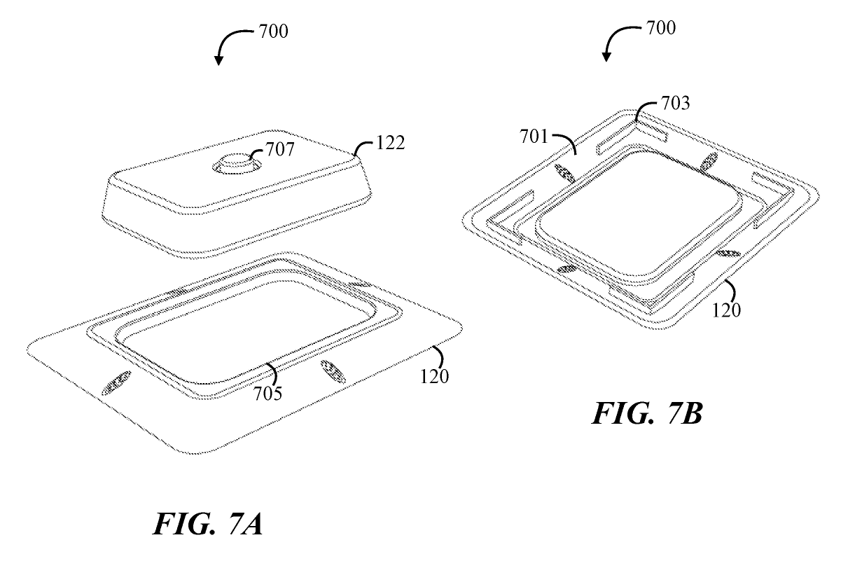

In reference to FIG. 7A and FIG. 7B, the top assembly 700 is illustrated according to some embodiments. The top assembly 700 is comprised of the weight 122 and the hive cover 120. The hive cover 120 should be dimensioned to rest atop the continuous top edge of the side walls 301, 303 and end walls 305, 307 to form a sufficiently sealed connection therebetween. FIG. 7B illustrates the bottom side 701 of the hive cover 120. Retaining members 703 may be positioned as protrusions to retain the hive cover 120 in position on top of the collapsible beehive assembly.

In some embodiments, the hive cover 120 has a recessed portion 705 to receive and retain the weight 122 thereon. To properly observe and maintain the colony within the beehive, the hive cover 120 is removable such that the beekeeper may open and close the collapsible beehive assembly when needed.

In some embodiments, the weight 122 has at least one aperture and closure 707 to permit the ingress and egress of a heavy material such as water or sand to provide suitable mass to the weight 122. The ability for the user to modulate the heaviness of the weight 122 allows for the user to empty the weight 122 during storage to ease transportation when not in use.

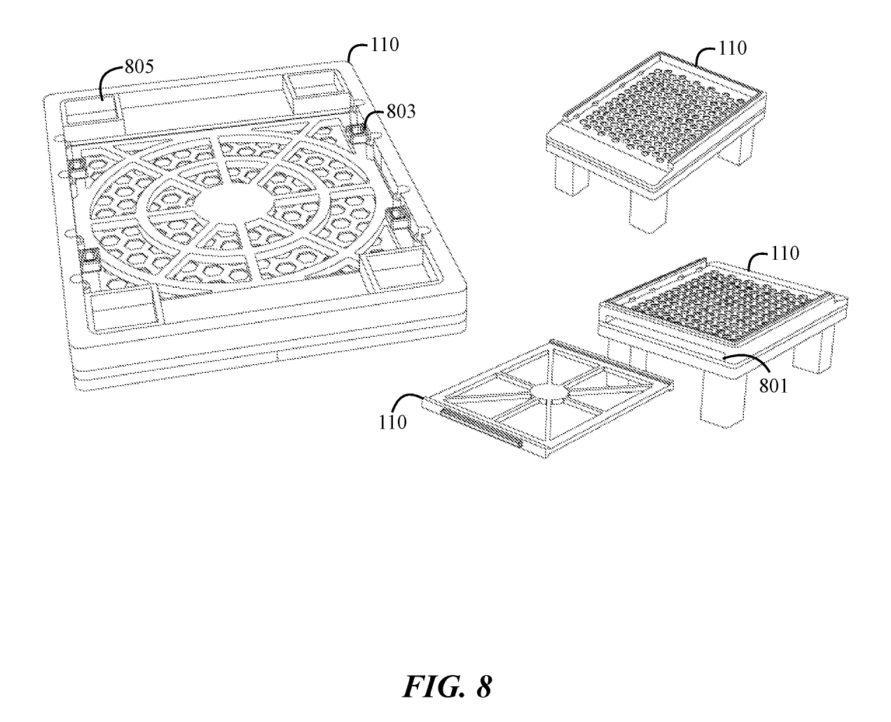

FIG. 8 illustrates the varroa monitoring tray 112 and bottom assembly 110 according to some embodiments. The bottom assembly 110 may be comprised of any assembly known in the arts. In the specific embodiment, a varroa monitoring tray opening 801 permits the varroa monitoring tray 112 to be disposed between the top and bottom components of the bottom board assembly. The bottom assembly 110 can include securing means 803 such as regions for tie downs as known in the arts. Legs are received by the leg receivers 805 to retain the legs during use.

An exemplary method of use is provided. During use, a user removes an optional buckle and strap and removes the hive cover from the collapsible beehive assembly. The inner cover is then removed from the hive allowing for the removal of the frames. Each collapsible box can then be removed following the removal of a crate support. During operation, the pivoting means may be locked into place. During deconstruction the pivoting means are unlocked and the insulation component is removed. The collapsible box can then be collapsed to reduce the overall volume. Each collapsible box is stored in the storage configuration as shown as described above. This process is repeated for each collapsible box stacked upon one another. The bottom assembly is then stored.

In some embodiments, the collapsible beehive assembly is designed to have a consistent bee spacing of 5-9 mm depending on the location to suit the particular bee species. The hive opening is 5/16 inches high to permit the ingress and egress of bees. The inner cover can include a vent opening of 1 1/18 by 3¾ inch slow for accessing the hive or feeding therethrough. If the hive requires better ventilation, or the bees require a second entrance at the top of the hive, the hive cover can be positioned to allow the additional entrance.

In some embodiments, the bottom assembly includes a closed bottom unlike a typical beehive, therefor, no mite board is needed.

In some embodiments, a foam tape can be attached over any aperture of the sidewalls or end walls to windproof the collapsible beehive assembly.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A collapsible beehive assembly comprising:
a bottom assembly having a plurality of collapsible boxes stacked thereon, each of the plurality of collapsible boxes configured to house a plurality of beehive frames, each of the plurality of collapsible boxes configured to receive one or more insulation components to winterize a collapsible beehive assembly;
an inner cover positioned on top of the plurality of collapsible boxes;
a hive cover positioned on top of the inner cover to enclose the beehive frames within the collapsible boxes; and
a weight positioned on top of the hive cover to retain the hive cover in a suitable position.

2. The assembly of claim 1, wherein each of the collapsible boxes is comprised of the following:
a first sidewall and a second sidewall, each sidewall including a handle portion to facilitate carrying of each of the collapsible boxes; and
a first end wall and a second end wall, each end wall including a pivoting means permitting the folding of each of the end walls to a storage configuration.

3. The assembly of claim 2, wherein each sidewall and each end wall are impermeable.

4. The assembly of claim 2, wherein the storage configuration reduces a storage volume to between a tenth and a twentieth in relation to an in-use volume.

5. The assembly of claim 1, wherein each of the sidewalls and each of the end walls slidingly engage with the one or more insulation components.

6. The assembly of claim 5, wherein the one or more insulation components are selected from the group consisting of Styrofoam, and tar paper.

7. The assembly of claim 1, wherein the weight is comprised of an aperture and a closure wherein materials are disposed.

8. The assembly of claim 7, wherein the hive cover is comprised of a recessed portion dimensioned to receive and retain the weight.

9. The assembly of claim 8, wherein the materials are water or sand.

10. The assembly of claim 1, wherein the bottom assembly is further comprised of a varroa monitoring tray disposed within a varroa monitoring tray opening of the bottom assembly.

11. A collapsible beehive assembly comprising:
a bottom assembly having a plurality of collapsible boxes stacked thereon, each of the plurality of collapsible boxes including a first and second sidewall and a first and second end wall arranged to form a rectangle to facilitate the cultivation of a beehive, each of the plurality of collapsible boxes configured to selectively receive one or more insulation components to winterize a collapsible beehive assembly;
an inner cover positioned on top and sealed to a top-most collapsible box;
a hive cover positioned on top of the inner cover to enclose the beehive frames within the collapsible boxes; and
a weight positioned on top of the hive cover to retain the hive cover in a suitable position, the weight retained in the suitable position by a recessed portion on the hive cover.

12. The assembly of claim 11, wherein the first and second sidewalls or the first and second end walls are comprised of a pivoting means permitting the folding of each of the end walls to a storage configuration.

13. The assembly of claim 11, wherein the first and second sidewalls and the first and second end walls are impermeable.

14. The assembly of claim 11, wherein the storage configuration reduces a storage volume to between a tenth and a twentieth in relation to an in-use volume.

15. The assembly of claim 11, wherein the first and second sidewalls and the first and second end walls slidingly engage with the one or more insulation components.

16. The assembly of claim 15, wherein the one or more insulation components are selected from the group consisting of styrofoam, and tar paper.

17. The assembly of claim 11, wherein the weight is comprised of an aperture and a closure wherein materials are disposed.

18. The assembly of claim 17, wherein the materials are water or sand.

19. The assembly of claim 11, wherein the bottom assembly is further comprised of a varroa monitoring tray disposed within a varroa monitoring tray opening of the bottom assembly.
